(19) United States
(12) Patent Application Publication
(10) Pub. No.: US 2025/0163142 A1
(43) Pub. Date: May 22, 2025

(54) FORMULATIONS OF MONOCLONAL ANTIBODIES

(71) Applicant: MedImmune Limited, Cambridge (GB)

(72) Inventors: Brian Lobo, Gaithersburg, MD (US); Deborah Sweet Goldberg, Gaithersburg, MD (US); Monika Sood Sharma, Gaithersburg, MD (US); Ambarish Umakant Shah, Gaithersburg, MD (US)

(21) Appl. No.: 18/676,425

(22) Filed: May 28, 2024

Related U.S. Application Data
(63) Continuation of application No. 17/224,900, filed on Apr. 7, 2021, now abandoned.
Application No. 17/224,900 is a continuation of application No. 16/488,302, filed on Aug. 23, 2019, now abandoned.
(60) Provisional application No. 62/465,269, filed on Mar. 1, 2017.

(51) Int. Cl. C07K 16/24; A61K 9/19; A61K 39/00; A61K 47/26; C07K 16/18

(52) U.S. Cl. CPC C07K 16/244; A61K 47/26; C07K 16/18; A61K 9/19; A61K 2039/545; C07K 2317/565; C07K 2317/94

(57) ABSTRACT

The present invention provides a formulation comprising: (i) a monoclonal antibody; and (ii) an ionic excipient; wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 6.5.

### CROSS-REFERENCE TO RELATED APPLICATIONS

This application is a continuation of U.S. patent application Ser. No. 17/224,900, filed Apr. 7, 2021, which is a continuation of U.S. patent application Ser. No. 16/488,302, filed Aug. 23, 2019, now abandoned, which is a 371 application of PCT/EP2018/054960, filed Feb. 28, 2018, which claims priority to, and the benefit of, U.S. Provisional Application No. 62/465,269, filed on Mar. 1, 2017, the contents of each of which is hereby incorporated by reference in its entirety.

### FIELD OF THE INVENTION

The invention is concerned with an antibody formulation, in particular, a monoclonal antibody formulation and uses thereof. The invention is particularly concerned with providing improved colloidal stability in an antibody formulation.

### SEQUENCE LISTING

The present specification makes reference to a Sequence Listing (submitted electronically as an .xml file named 20240528 KPL-036US3 SL.xml on May 28, 2024). The xml file was generated on May 28, 2024 and is 23 KB in size. The entire contents of the sequences are herein incorporated by reference.

### BACKGROUND OF THE INVENTION

As a result of the isoelectric point (pI) of a number of monoclonal antibodies being in the preferred pharmaceutical pH formulation range for proteins (pH 5.5 to pH 7.5), these molecules present unique formulation challenges.

Colloidal instability at a molecule's pI is due to a lack of an electrostatic charge on the molecule, which allows closer protein-protein interactions (so-called “self-association”) that lead to physical instabilities. For this reason, the pH of a protein formulation is typically selected to be at least 1 pH unit away from the protein pI. This aims to provide colloidal stability and thus prevent physical instabilities, such as aggregation, precipitation, opalescence, phase separation and/or particle formation.

According to the ‘1 pH unit away’ rule, antibodies having a low or neutral pI e.g. a pI of pH 5.5 to pH 7.5 thus should be formulated into a formulation with a pH outside the range of 5.5 to 7.5. However, outside this range, additional instabilities can be observed. At more acidic pH, an increased rate of fragmentation, reduced conformational stability and increased aggregation can be observed. At more basic pH, the potential for increased oxidation, deamidation and fragmentation and incompatibility with glass containers are present.

The above instabilities are particularly problematic in such antibody formulations where the antibody is present at a commercially desirable concentration e.g. 50 mg/ml and above.

Therefore, there exists a need to provide an improved formulation for an antibody having a low or neutral pI. In particular, there exists a need to provide a stable formulation for an antibody having a low or neutral pI and, particularly such a formulation having a commercially desirable antibody concentration.

### BRIEF SUMMARY OF THE INVENTION

The present invention provides a new antibody formulation, in particular a new monoclonal antibody formulation. In particular, the present formulation provides a means for improving colloidal stability for antibodies having a low or neutral pI. The present invention thus provides an alternative to the ‘1 pH away’ rule for providing colloidal stability. The present invention thus allows antibodies having a low or neutral pI to be formulated within 1 pH unit of the antibody pI. Thus, the present invention enables such antibodies to be formulated within a pH range of 5.5 to 7.5 and at a commercially useful concentration, whilst substantially avoiding the instabilities associated with more acidic or more basic pHs.

The invention provides a formulation comprising:
i. a monoclonal antibody; and
ii. an ionic excipient;
   wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5.

The invention thus further provides a formulation comprising:
i. a monoclonal antibody; and
ii. an ionic excipient;
   wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5; and wherein the aggregation rate of the monoclonal antibody in the formulation is reduced compared to the aggregation rate of the same antibody in the same formulation but without an ionic excipient.

The formulations of the present invention are particularly useful for antibodies having a low or neutral pI, for example in the range of pH 5.5 to pH 7.5. The invention provides a formulation comprising:
i. a monoclonal antibody having a low or neutral pI (e.g. 5.5 to 7.5); and
ii. an ionic excipient;
   wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5.

The invention thus further provides a formulation comprising:
i. a monoclonal antibody having a low or neutral pI (e.g. 5.5 to 7.5); and
ii. an ionic excipient;
   wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5; and wherein the aggregation rate of the monoclonal antibody in the formulation is reduced compared to the aggregation rate of the same antibody formulated in the same formulation but without an ionic excipient.

In one embodiment, the monoclonal antibody has a pI in the range of 5.5 to 7.5. In one embodiment, the monoclonal antibody has a pI in the range of 6.0 to 7.5. In one embodiment, the monoclonal antibody has a pI in the range of 6.3 to 7.5. In one embodiment, the monoclonal antibody has a pI in the range of 6.4 to 7.5. Without wishing to be bound by theory, a low to neutral pI can occur for proteins where there is a either a net balance of oppositely charged (positive amine groups and negative carboxylate groups) amino acid side chains on the protein or different domains have overall opposite charge, within a pH range of 5.5 to 7.5. Again, without wishing to be bound by theory, it is possible that the ionic excipient in the formulation of the invention shields these opposing and attractive charges, thus colloidally stabilizing proteins having a pI within this range. The present invention thus provides use of an ionic excipient in an antibody formulation for the purpose of changing the charge state or distribution of the antibody in the formulation. The present invention further provides use of an ionic excipient in an antibody formulation for the purpose of colloidally stabilizing the antibody in the formulation.

In one embodiment, the monoclonal antibody is present in the formulations described herein at a concentration of about 75 mg/ml or greater (e.g. about 75 mg/ml to about 200 mg/ml). In one embodiment, the monoclonal antibody is present in the formulations described herein at a concentration of about 100 mg/ml or greater (e.g. about 100 mg/ml to about 200 mg/ml). In one embodiment, the monoclonal antibody is present in the formulations described herein at a concentration of about 100 mg/ml to about 165 mg/ml.

In one embodiment, the monoclonal antibody is present at a concentration of about 100 mg/ml.

In one embodiment, the ionic excipient is present at a concentration of about 75 mM to about 100 mM. In one embodiment, the ionic excipient is present at a concentration of about 75 mM. In one embodiment, the ionic excipient is present at a concentration of about 80 mM.

In one embodiment, the monoclonal antibody is an IgG1 or IgG4 monoclonal antibody. Most preferably, the monoclonal antibody is an IgG4 monoclonal antibody. IgG4 antibodies typically have a low or neutral pI. The invention thus provides a formulation comprising:
i. an IgG4 monoclonal antibody; and
ii. an ionic excipient;
   wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5.

The invention thus further provides a formulation comprising:
i. an IgG4 monoclonal antibody; and
ii. an ionic excipient;
   wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5; and wherein the aggregation rate of the monoclonal antibody in the formulation is reduced compared to the aggregation rate of the same antibody in the same formulation but without an ionic excipient.

In one embodiment, the formulations described herein have a pH in the range of about pH 5.5 to about pH 6.5. In one embodiment, the formulations described herein have a pH in the range of about pH 5.7 to about pH 6.3. In one embodiment, the formulations described herein have a pH in the range of about pH 5.7 to about pH 6.1. Preferred formulations have a pH of about 5.8. Other preferred formulations have a pH of about 6.0.

In one embodiment, the ionic excipient is a charged amino acid. In one embodiment, the ionic excipient is lysine. In another embodiment, the ionic excipient is arginine.

In one embodiment, the ionic excipient is a salt. The invention thus provides a formulation comprising:
i. a monoclonal antibody as defined anywhere herein; and
ii. a salt;
   wherein the monoclonal antibody is present at a concentration of about 50 mg/ml or greater (e.g. about 50 mg/ml to about 200 mg/ml) and the salt is present at a concentration of about 50 to about 150 mM and the formulation has a pH of 5.5 to 7.5.

The invention thus further provides a formulation comprising:
i. a monoclonal antibody as defined anywhere herein; and
ii. a salt;
   wherein the monoclonal antibody is present at a concentration of about 50 mg/ml or greater (e.g. about 50 mg/ml to about 200 mg/ml) and the salt is present at a concentration of about 50 to about 150 mM and the formulation has a pH of 5.5 to 7.5; and wherein the aggregation rate of the monoclonal antibody in the formulation is reduced compared to the aggregation rate of the same antibody in the same formulation but without a salt.

In one embodiment, the salt is present at a concentration of about 75 mM to about 100 mM. In one embodiment, the salt is present at a concentration of about 75 mM or about 80 mM.

In one embodiment, the salt is NaCl, for example at a concentration of about 75 mM to about 100 mM, suitably at a concentration of about 75 mM.

In one embodiment, the salt is arginine hydrochloride, for example at a concentration of about 75 mM to about 100 mM, suitably at a concentration of about 80 mM.

In one embodiment, the formulation further comprises a sugar. Amongst other known benefits, the presence of a sugar can improve tonicity of the formulation. This is desirable since preferred formulations are isotonic or near isotonic (for example, having an osmolality between 240 to 500 mOsm/kg. In one embodiment, the ionic excipient is a salt and the formulation further comprises a sugar.

In one embodiment, the formulation further comprises a sugar and the ionic excipient is present at a concentration in the range of about 75 mM to about 150 mM. In one embodiment, the formulation further comprises a sugar and the ionic excipient is present at a concentration in the range of about 75 mM to about 100 mM. In one embodiment, the formulation further comprises a sugar, which is present at a concentration in the range of about 100 mM to 140 mM, and the ionic excipient is present at a concentration in the range of about 75 mM to 100 mM.

The invention thus provides a formulation comprising:
i. a monoclonal antibody as defined anywhere herein;
ii. an ionic excipient (e.g. a salt) as defined anywhere herein;
iii. a sugar as defined anywhere herein; and
   wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5. The ionic excipient preferably is present at a concentration in the range of about 75 mM to about 150 mM, more preferably about 75 mM to about 100 mM.

The invention thus further provides a formulation comprising:
i. a monoclonal antibody as defined anywhere herein; and
ii. an ionic excipient (e.g. a salt) as defined anywhere herein;
iii. a sugar as defined anywhere herein; and
   wherein the monoclonal antibody is present at a concentration of about 50 mg/ml or greater (e.g. about 50 mg/ml to about 200 mg/ml) and the ionic excipient is present at a concentration of about 50 of about 150 mM and the formulation has a pH of 5.5 to 7.5; and wherein the aggregation rate of the monoclonal antibody in the formulation is reduced compared to the aggregation rate of the same antibody in the same formulation but without an ionic excipient. The ionic excipient preferably is present at a concentration in the range of about 75 mM to about 150 mM, more preferably about 75 mM to about 100 mM.

In one embodiment, the sugar is trehalose. In another embodiment, the sugar is sucrose. For example, the sugar is found at a concentration of about 100 mM to about 140 mM, suitably at a concentration of about 120 mM.

In one embodiment, the formulation further comprises one or more buffers. In one embodiment, the one or more buffers is a buffer comprising histidine. In one embodiment, the one or more buffers are selected from a buffer comprising histidine succinate, histidine acetate, histidine citrate, histidine chloride or histidine sulfate. In one embodiment, the one or more buffers is histidine, histidine hydrochloride, or a combination thereof (histidine/histidine hydrochloride). In one embodiment, the one or more buffers is L-histidine/L-histidine hydrochloride monohydrate. For example, the buffer may be at a concentration of about 10 mM to about 50 mM, suitably at a concentration of about 30 mM. It will be understood that a buffer may, itself, be an ionic excipient. Thus, in one embodiment, the buffer is the ionic excipient. In this embodiment, the concentration of the buffer should be above 50 mM i.e. in line with the concentration of the ionic excipient disclosed herein. Put another way, in one embodiment, the ionic excipient also acts as a buffer in the formulation. In this embodiment, an additional buffer may or may not be present.

In one embodiment, the formulation further comprises a surfactant. In one embodiment, the surfactant is a polysorbate, including for example, polysorbate-80.

In one embodiment, the formulation further comprises a sugar and one or more buffers. In one embodiment, the ionic excipient is a salt and the formulation further comprises a sugar and one or more buffers.

In one embodiment, the formulation further comprises a surfactant, a sugar and one or more buffers. In one embodiment, the ionic excipient is a salt and the formulation further comprises a surfactant, a sugar and one or more buffers.

The invention thus provides a formulation comprising:
i. a monoclonal antibody as defined anywhere herein;
ii. an ionic excipient (e.g. a salt) as defined anywhere herein;
iii. a sugar as defined anywhere herein;
iv. one or more buffers as defined anywhere herein; and
v. optionally a surfactant as defined anywhere herein
   wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5.

The invention thus further provides a formulation comprising:
i. a monoclonal antibody as defined anywhere herein; and
ii. an ionic excipient (e.g. a salt) as defined anywhere herein;
iii. a sugar as defined anywhere herein;
iv. one or more buffers as defined anywhere herein; and
v. optionally a surfactant as defined anywhere herein
   wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5; and wherein the aggregation rate of the monoclonal antibody in the formulation is reduced compared to the aggregation rate of the same antibody in the same formulation but without an ionic excipient.

The formulations described herein can also include one or more additional excipients, including for example, one or more sugars, salts, amino acids, polyols, chelating agents, emulsifiers and/or preservatives.

One preferred formulation provided by the invention comprises 150 mg/ml antibody, 50 mM sodium acetate/acetic acid, 106 mM trehalose dehydrate, 70 mM sodium chloride, 0.05% (w/v) Polysorbate 80, wherein the formulation has a pH of pH 5.8. The antibody in this formulation is preferably an anti-GM-CSF-Rα IgG4 antibody, more preferably an anti-GM-CSF-Rα IgG4 antibody having the VH and VL sequences of the CAM-3001 antibody as described herein. The antibody in this formulation is preferably an anti-GM-CSF-Rα IgG4 antibody having the same 6 CDRs as the CAM-3001 antibody as described herein.

One preferred formulation provided by the invention comprises 150 mg/ml antibody, 50 mM Sodium Acetate, 85 mM sodium chloride and 0.01% Polysorbate 80, wherein the formulation has a pH of pH 5.5. The antibody in this formulation is preferably an anti-IL-13 IgG4 antibody, more preferably an anti-IL-13 IgG4 antibody having the VH and VL sequences of the CAT-354 antibody as described herein. The antibody in this formulation is preferably an anti-IL-13 IgG4 antibody having the same 6 CDRs as the CAT-354 antibody as described herein.

The formulations of the invention preferably are pharmaceutical formulations.

The present invention provides a pharmaceutical formulation as described anywhere herein for use as a medicament.

The present invention provides a pharmaceutical formulation as described anywhere herein for use in the treatment of a disease.

The present invention provides a method of treating a disease in a subject comprising administering a pharmaceutical formulation as described anywhere herein to the subject.

Also provided herein are methods of treating a subject by administering a therapeutically effective amount of a pharmaceutical formulation as described anywhere herein to the subject.

In one embodiment, the subject is a human.

The disease to be treated will be dependent on the particular antibody contained in the formulation. In one embodiment, the disease is cancer. The disease may be selected from the group consisting of diabetes, cardiovascular diseases, infectious disease, rheumatoid arthritis, vasculitis, giant cell arthritis, glomerular nephropathy, lupus nephritis, uveitis, atopic dermatitis, cirrhosis, psoriatic arthritis, chronic obstructive pulmonary disease, severe asthma, neutrophilic asthma, and myeloid leukemia.

The present invention provides a lyophilized cake capable of being reconstituted using only sterile water into a formulation as defined herein or a pharmaceutical formulation as defined herein. Also provided herein is a formulation capable of being lyophilised to form a lyophilized cake, wherein the lyophilized cake is capable of being reconstituted using only sterile water into a formulation as defined herein or a pharmaceutical formulation as defined herein.

### BRIEF DESCRIPTIONS OF THE DRAWINGS

FIG. 1A shows the conversion of an IgG4 monoclonal antibody to a half molecule.

FIG. 1B and FIG. 1C show sequences relating to the CAT-354 antibody. The V_{H} and V_{L} sequences are labelled and the 6 CDRs are underlined.

FIG. 2 shows the effect of salt on the aggregation rate of IgG4 molecules.

FIG. 3A shows the results of varying pH on k_{D}.

FIG. 3B shows the results of varying pH on Kd in the presence of 100 mM salt.

FIG. 4 shows the effect of salt on k_{D}. Note generally a reduction in colloidal stability at pH 5, but an increase in colloidal stability at pH 6 and pH 7.

FIG. 5 shows aggregation rate data at 40° C. for formulations with or without 100 mM salt.

FIG. 6 shows change in aggregation rate with formulation pH for MEDI7814.

FIG. 7 shows the impact of ionic excipients on antibody aggregation rate at pH6 (MEDI7814).

FIG. 8 shows the MEDI8897 heavy chain nucleotide sequence and translation. CDRs are underlined, amino acid differences form allelic constant regions have been circled and division between the variable and constant regions marked by a ‘ ’).

FIG. 9 shows the MEDI8897 light chain nucleotide sequence and translation. CDRs are underlined and division between the variable and constant regions marked by a ‘ ’).

FIG. 10 shows MEDI8897 formulation stability over 3 month period at 5° C., 25° C. and 40° C.

FIG. 11 shows the impact of ionic excipients on antibody aggregation rate at pH 6 (MEDI578).

FIG. 12 shows purity by size-exclusion chromatography.

FIG. 13 shows a reduction in subvisible particles in formulations containing ionic excipients under thermal stress conditions. Colloidal stability is improved.

FIG. 14 shows a comparison table of control of test buffer system.

### DETAILED DESCRIPTION OF THE INVENTION

Due to the fact that a number of monoclonal antibodies, and in particular IgG4 antibodies, possess a pI that is close to physiologic pH, i.e. the pH generally desired for human administration, difficulties in formulating these monoclonal antibodies occur. These difficulties can relate particularly to colloidal stability.

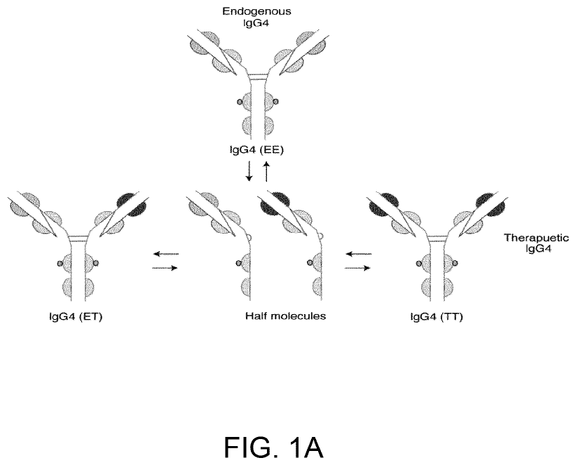

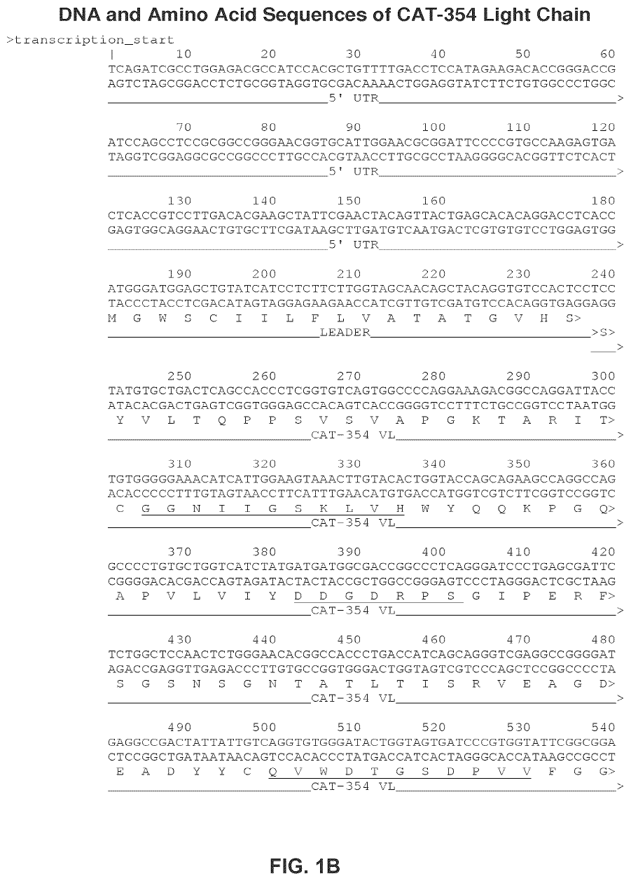

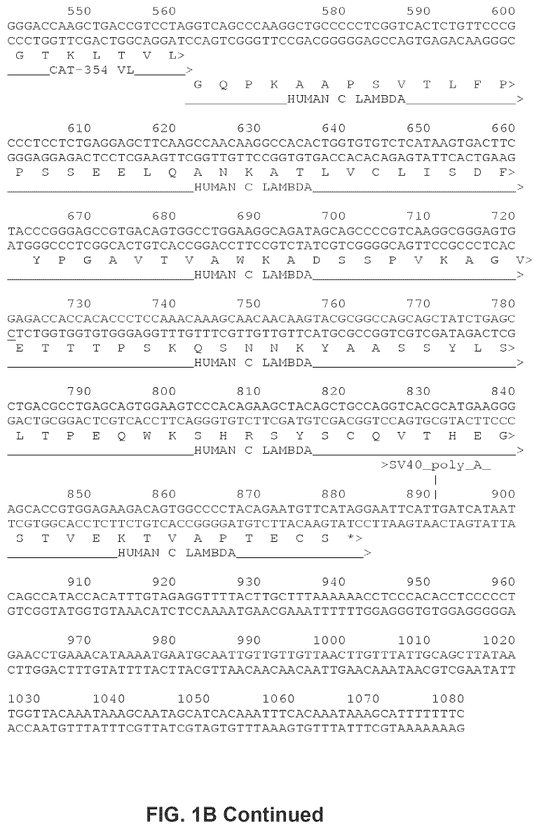

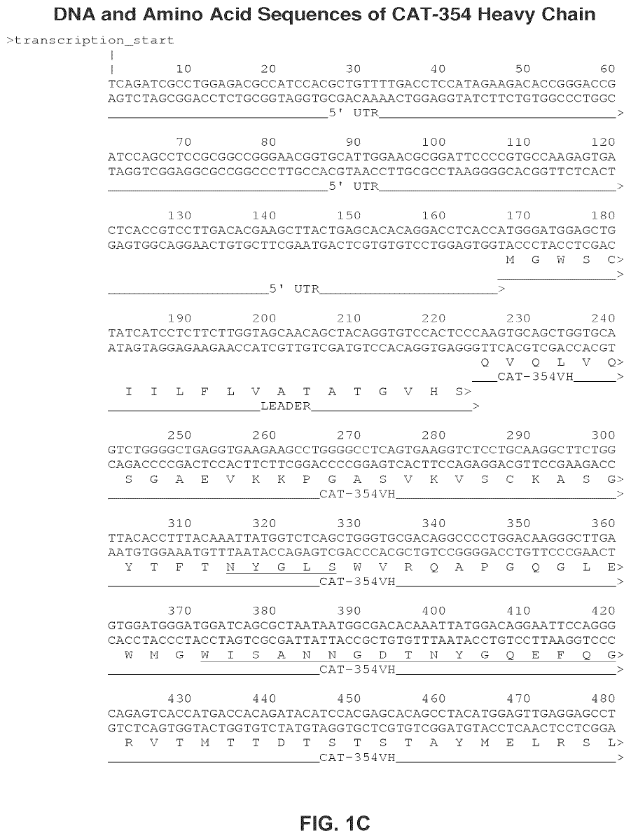

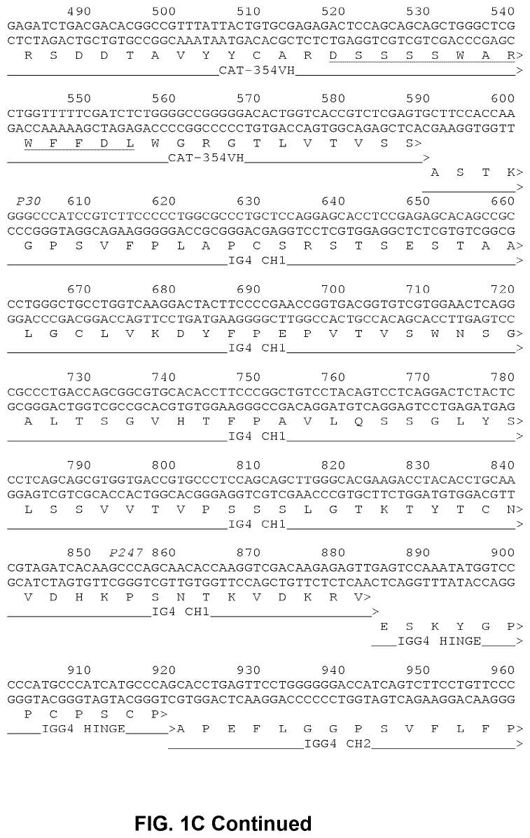

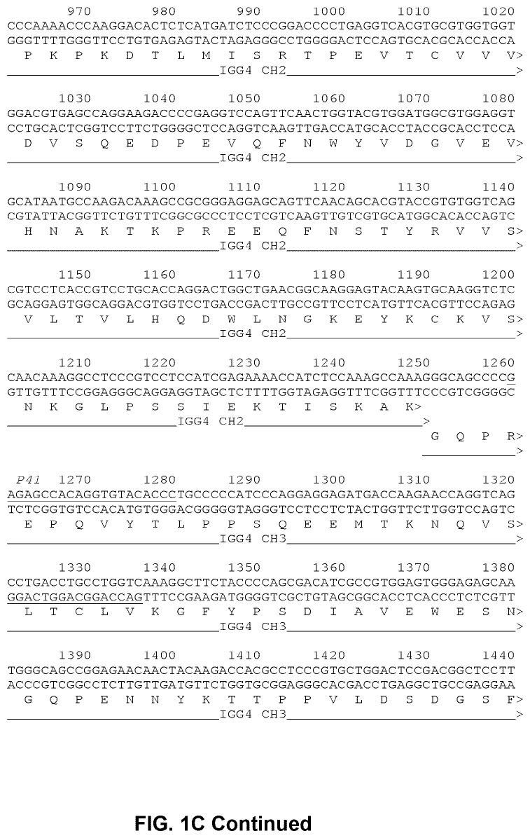

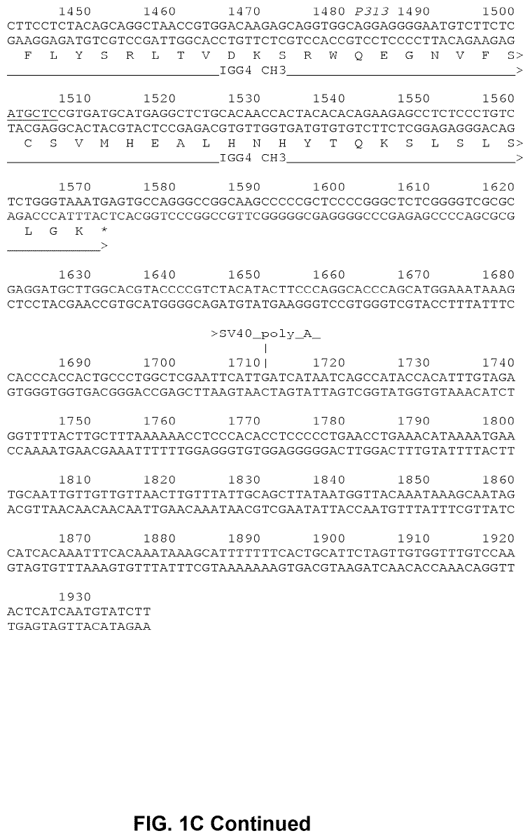

IgG4 monoclonal antibodies are characterized by a structure which prevents complement activation, and also allows for an in vivo half molecular exchange (one heavy and one light chain) between two IgG4 molecules, generating a new IgG4 with a bivalent reactivity. See FIG. 1A. The IgG4 hinge is three amino acids shorter than the hinge of IgG1, and IgG4 has two cysteines that are available for the covalent interaction between the H-chains. [Aalberse and Schuurman, “IgG4 breaking the rules,” Immunology 2002 105:9-19].

The present invention provides a new monoclonal antibody formulation, in particular a new IgG4 monoclonal antibody formulation and a new IgG1 monoclonal antibody formulation.

The invention provides a formulation comprising: (i) a monoclonal antibody; and (ii) an ionic excipient (e.g. a salt); wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5.

The invention further provides a formulation comprising: (i) a monoclonal antibody; and (ii) an ionic excipient (e.g. a salt); wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5; and wherein the aggregation rate of the monoclonal antibody in the formulation is reduced compared to the aggregation rate of the same antibody in the same formulation but without an ionic excipient.

Aggregation rate can be measured according to standard techniques as described herein. Surprisingly, formulations in accordance with the present invention have been shown to have good stability and to have decreased self-aggregation e.g. to exhibit ≤2.0% aggregation when stored at room temperature for 3 months. The present invention thus provides the use of an ionic excipient in an antibody formulation for the purpose of increasing stability of the antibody in the formulation. The present invention further provides the use of an ionic excipient in an antibody formulation for the purpose of decreasing self-aggregation of the antibody in the formulation.

The formulations of the present invention are particularly useful for antibodies having a low or neutral pI, for example antibodies that have a pI in the range pH 5.5 to pH 7.5, pH 6.0 to pH 7.5, pH 6.3 to pH 7.5, pH 6.4 to pH 7.5, or pH 6.5 to pH 7.5. The pI of an antibody can be measured according to standard techniques, for example by capillary isoelectric focusing (cIEF). The invention thus provides a formulation comprising: (i) a monoclonal antibody having a low or neutral pI (e.g. 5.5 to 7.5); and (ii) an ionic excipient; wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5. The invention thus further provides a formulation comprising: (i) a monoclonal antibody having a low or neutral pI (e.g. 5.5 to 7.5); and (ii) an ionic excipient; wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5; and wherein the aggregation rate of the monoclonal antibody in the formulation is reduced compared to the aggregation rate of the same antibody in the same formulation but without an ionic excipient.

In one embodiment, the monoclonal antibodies have a pI in the range of pH 6.4 to pH 7.5. In another embodiment, the monoclonal antibody described herein has a pI in the range of about pH 5.5 to about pH 6.0, about pH 5.7 to about pH 6.0, or about pH 5.5, about pH 5.6, about pH 5.7, about pH 5.8, about pH 5.9, about pH 6.0, about pH 6.1, about pH 6.2, about pH 6.3, about pH 6.4, or about pH 6.5. In embodiments, the pI of the monoclonal antibodies provided herein is 5.7 to 6.0, more suitably a pI of about 5.8 or 6.0.

In one embodiment, the monoclonal antibody is an IgG1 or IgG4 monoclonal antibody. Most preferably, the monoclonal antibody is an IgG4 monoclonal antibody. The invention thus provides a formulation comprising: (i) an IgG4 monoclonal antibody having a low or neutral pI (e.g. 5.5 to 7.5); and (ii) an ionic excipient; wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5. The invention thus further provides a formulation comprising: (i) an IgG4 monoclonal antibody having a low or neutral pI (e.g. 5.5 to 7.5); and (ii) an ionic excipient; wherein the monoclonal antibody is present at a concentration of about 50 mg/ml or greater (e.g. about 50 mg/ml to about 200 mg/ml) and the ionic excipient is present at a concentration of about 50 to about 150 mM and the formulation has a pH of 5.5 to 7.5; and wherein the aggregation rate of the monoclonal antibody in the formulation is reduced compared to the aggregation rate of the same antibody in the same formulation but without an ionic excipient.

In one embodiment, the monoclonal antibody is an anti-GM-CSF-Rα monoclonal antibody, an anti-IL-13 monoclonal antibody, an anti-RSV monoclonal antibody, or an anti-C5/C5a monoclonal antibody. In exemplary embodiments, the monoclonal antibody is an IgG4 monoclonal antibody, for example, an anti-GM-CSF-Rα monoclonal antibody. In one embodiment, the monoclonal antibody is CAM-3001. In one embodiment, the monoclonal antibody is MEDI8897.

Monoclonal antibodies include antibody functional parts, e.g., antibodies or antigen-binding fragments, variants, or derivatives thereof. Monoclonal antibodies further include, but are not limited to, human, humanized, or chimeric antibodies, single chain antibodies, bispecific antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule. In a preferred embodiment, the monoclonal antibody is an IgG1 or IgG4 monoclonal antibody.

### Antibody Concentration

Suitably, the monoclonal antibody is present in the formulations described herein at a commercially desirable concentration e.g. of about 50 mg/ml to about 300 mg/ml, about 50 mg/ml to about 200 mg/ml, about 100 mg/ml to about 200 mg/ml, about 100 mg/ml to about 165 mg/ml, about 100 mg/ml to about 150 mg/ml, or about 50 mg/ml, about 75 mg/ml, about 100 mg/ml, about 105 mg/ml, about 110 mg/ml, about 115 mg/ml, about 120 mg/ml, about 125 mg/ml, about 130 mg/ml, about 135 mg/ml, about 140 mg/ml, about 145 mg/ml, about 150 mg/ml, about 155 mg/ml, about 160 mg/ml, about 165 mg/ml, about 170 mg/ml, about 175 mg/ml, about 180 mg/ml, about 185 mg/ml, about 190 mg/ml, about 195 mg/ml, or about 200 mg/ml, including values and ranges within these ranges.

Suitably, the monoclonal antibody is present in the formulations described herein at a concentration of about 100 mg/ml to about 165 mg/ml. Suitably, the monoclonal antibody is present in the formulations described herein at a concentration of about 100 mg/ml.

pH

Suitably, the formulations described herein have a pH in the range of about pH 5.5 to about pH 7.5 in order to provide near optimal or optimal chemical stability (hydrolysis, deamidation, isomerization). In one embodiment, the formulations described herein have a pH in the range of about pH 5.7 to about pH 6.3. In one embodiment, the formulations described herein have a pH in the range of about 5.5 to about 6.5. In one embodiment, the formulations described herein have a pH in the range of about pH 5.7 to about pH 6.1. Preferred formulations have a pH of about 5.8. Other preferred formulations have a pH of about 6.0.

Suitably, the formulations described herein have a pH in the range of about pH 5.5 to about pH 6.0, about pH 5.7 to about pH 6.0, or about pH 5.5, about pH 5.6, about pH 5.7, about pH 5.8, about pH 5.9, about pH 6.0, about pH 6.1, about pH 6.2, about pH 6.3, about pH 6.4, or about pH 6.5. In embodiments, the pH of the formulations provided herein is 5.7 to 6.0, more suitably the formulations have a pH of about 5.8 or 6.0.

A formulation pH close to about pH 7.4 also can be desirable for injection site tolerability.

### Ionic Excipient

Exemplary ionic excipients for use in the formulations include salts and charged amino acids. The ionic excipient might comprise a combination of a salt and charged amino acid.

Exemplary charged amino acids include arginine and lysine.

Exemplary salts include salts of charged amino acids, for example, succinate, acetate, and sulfate salts of arginine and lysine.

Further exemplary salts are those described herein including, but not limited to, sodium chloride, as well as other salts with sodium, potassium, calcium, magnesium and the like, such as chlorides, carbonates, sulphates, acetates, gluconates, lactates, malates, and other auxiliaries and the like which are customary in the field of parenteral administration.

Suitably the salt is selected from sodium chloride (NaCl), lysine hydrochloride and arginine hydrochloride. In one embodiment, the salt is NaCl. In another embodiment, the salt is arginine hydrochloride. In another embodiment, the salt is lysine hydrochloride.

The concentration of the ionic excipient, suitably salt, in the pharmaceutical formulations described herein is generally in the range of about 50 mM to about 150 mM, more suitably about 50 mM to about 100 mM, about 60 mM to about 80 mM, or about 50 mM, about 55 mM, about 60 mM, about 65 mM, about 70 mM, about 75 mM, about 80 mM, about 85 mM, about 90 mM, about 95 mM or about 100 mM, including any ranges or values within these ranges.

In one embodiment, the ionic excipient is present at a concentration of about 50 mM to about 125 mM.

In one embodiment, the ionic excipient is present at a concentration of about 50 mM to about 100 mM.

In one embodiment, the ionic excipient is present at a concentration of about 75 mM to about 100 mM.

In suitable embodiments, the salt is NaCl, for example at a concentration of about 50 mM to about 100 mM, suitably at a concentration of about 70 mM.

In suitable embodiments, the salt is arginine hydrochloride, for example at a concentration of about 50 mM to about 100 mM, suitably at a concentration of about 80 mM.

### Buffers

The formulations described herein suitably comprise one or more buffers. As used herein, “buffer” refers to an excipient for maintaining the pH of a formulation. Exemplary buffers for use in the formulations provided herein include, but are not limited to histidine, histidine hydrochloride (histidine HCl), sodium succinate, sodium acetate, sodium acetate/acetic acid, sodium phosphate, citrate, phosphate, succinate, glycine, and acetate. In one embodiment, the buffer for use in the formulations described herein is sodium acetate/acetic acid. In one embodiment, the one or more buffers is a buffer comprising histidine. In one embodiment, the one or more buffers are selected from a buffer comprising histidine succinate, histidine acetate, histidine citrate, histidine chloride or histidine sulfate. In one embodiment, the one or more buffers is histidine, histidine hydrochloride, or a combination thereof (histidine/histidine hydrochloride). In one embodiment, the one or more buffers is L-histidine/L-histidine hydrochloride monohydrate.

The concentration of a buffer, suitably sodium acetate/acetic acid, in the pharmaceutical formulations described herein is generally in the range of about 10 mM to about 100 mM, more suitably about 15 mM to about 80 mM, about 25 mM to about 75 mM, about 30 mM to about 60 mM, about 40 mM to about 60 mM, about 40 mM to about 50 mM, or about 15 mM, about 20 mM, about 25 mM, about 30 mM, about 35 mM, about 40 mM, about 45 mM, about 50 mM, about 55 mM, about 60 mM, about 65 mM, about 70 mM or about 75 mM, including any ranges or values within these ranges.

In one embodiment, the one or more buffers is L-histidine/L-histidine hydrochloride monohydrate, for example at a concentration of about 10 mM to about 50 mM, suitably at a concentration of about 30 mM.

The pH of the buffer is preferably in the range of pH 5.5 to pH 6.0.

It will be understood that a buffer may, itself, be an ionic excipient. Thus, in one embodiment, the buffer is the ionic excipient. In this embodiment, the concentration of the buffer should be above 50 mM i.e. in line with the concentration of the ionic excipient disclosed herein. Preferable concentrations for the buffer in this embodiment are as discussed anywhere herein in relation to the ionic excipient.

Put another way, in one embodiment, the ionic excipient also acts as a buffer in the formulation. In this embodiment, an additional buffer may or may not be present.

### Sugars and Surfactants

The formulations described herein suitably comprise a sugar, for example, but not limited to, trehalose, lactose, mannitol, mellibiose, melezitose, raffinose, mannotriose, stachyose and sucrose. In other embodiments, a polyol such as trihydric or higher molecular weight sugar alcohols, e.g. glycerin, dextran, erythritol, glycerol, arabitol, xylitol, sorbitol, and mannitol, can be used. Examples of reducing sugars include, but are not limited to, glucose, maltose, maltulose, iso-maltulose and lactulose. Examples of non-reducing sugars include, but are not limited to, trehalose, non-reducing glycosides of polyhydroxy compounds selected from sugar alcohols and other straight chain polyalcohols. Examples of sugar alcohols include, but are not limited to, monoglycosides, compounds obtained by reduction of disaccharides such as lactose, maltose, lactulose and maltulose. The glycosidic side group can be either glucosidic or galactosidic. Additional examples of sugar alcohols include, but are not limited to, glucitol, maltitol, lactitol and iso-maltulose. In one embodiment, the sugar is selected from the group consisting of trehalose, lactose, mannitol, raffinose and sucrose. In specific embodiments, trehalose is used as a sugar in the formulations described herein. In specific embodiments, sucrose is used as a sugar in the formulations described herein.

Suitably, the amount of sugar, for example trehalose, in a formulation described herein is about 1% (w/v) to about 10% (w/v). Unless otherwise noted, percentage of a component (%) is used herein indicate a weight/volume (w/v) %. In exemplary embodiments, the amount of sugar in a pharmaceutical formulation described herein is about 1% (w/v) to about 8% (w/v), or about 2% (w/v) to about 6% (w/v), about 2% (w/v) to about 5% (w/v), about 3% (w/v) to about 5% (w/v), or about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v), including any values and ranges within these ranges.

The formulations described herein suitably comprise a surfactant.

The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical formulations and preparations of biological materials. Pharmaceutically acceptable surfactants like polysorbates (e.g. polysorbates 20, 40, 60 or 80); polyoxamers (e.g. poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N. J.), polyethyl glycol, polypropyl glycol, and copolymers of ethylene and propylene glycol (e.g. Pluronics, PF68 etc), can be used in the pharmaceutical formulations described herein. Suitably the surfactant is a polysorbate, including for example, polysorbate-20, polysorbate-40, polysorbate-60, and polysorbate-80. In one embodiment, the surfactant is polysorbate-80.

Suitably, the formulations described herein comprise a surfactant (suitably polysorbate-80) at about 0.001% to about 0.5% (w/v), more suitably about 0.002% to about 0.1% of a surfactant, for example about 0.01% to about 0.2%, about 0.02% to about 0.01%, about 0.02% to about 0.07%, about 0.03% to about 0.06%, about 0.04% to about 0.06%, or about 0.02%, about 0.025%, about 0.03%, about 0.035%, about 0.04%, about 0.045%, about 0.05%, about 0.055%, about 0.060%, about 0.065%, about 0.07%, about 0.075%, about 0.08%, about 0.085%, about 0.09%, about 0.095%, or about 0.1% of a surfactant, including any ranges or values within these ranges.

The formulations described herein suitably comprise a surfactant and a sugar. The formulations described herein suitably comprise a surfactant and one or more buffers. The formulations described herein suitably comprise a sugar and one or more buffers. The formulations described herein suitably comprise a surfactant, a sugar, and one or more buffers.

The formulations described herein can also include one or more additional excipients, including for example, one or more sugars, salts, amino acids, polyols, chelating agents, emulsifiers and/or preservatives.

### Pharmaceutical Use

The formulations of the invention preferably are pharmaceutical formulations. Suitably, the pharmaceutical formulations described herein are “pharmaceutically acceptable,” and thus would meet the necessary approval requirements required by a regulatory agency of the Federal or a state government, or listed in the U.S. Pharmacopeia, European Pharmacopeia, or other generally recognized pharmacopeia, so as to be used in animals, and more particularly in humans.

The present invention provides a pharmaceutical formulation as described anywhere herein for use as a medicament. The present invention provides a pharmaceutical formulation as described anywhere herein for use in the treatment of a disease. The present invention provides a method of treating a disease in a subject comprising administering a pharmaceutical formulation as described anywhere herein to the subject. Also provided herein are methods of treating a subject by administering a therapeutically effective amount of a pharmaceutical formulation as described anywhere herein to the subject.

As used herein, the term “subject” includes any human or nonhuman animal. The term “nonhuman animal” includes all vertebrates, for example, but not limited to, mammals and non-mammals, such as nonhuman primates, sheep, dogs, cats, horses, cows, chickens, amphibians, reptiles, etc. In one embodiment, the subject is a human.

The disease may be selected from the group consisting of diabetes, cardiovascular disease, infectious disease, rheumatoid arthritis, vasculitis, giant cell arthritis, glomerular nephropathy, lupus nephritis, uveitis, atopic dermatitis, cirrhosis, psoriatic arthritis, chronic obstructive pulmonary disease, severe asthma, neutrophilic asthma, and myeloid leukemia.

In embodiments, the formulation is administered to a subject subcutaneously or by injection.

Suitably, the formulations are a liquid formulation or a frozen formulation.

Also provided herein are methods of preparing a pharmaceutical formulation comprising preparing a pharmaceutical formulation as described herein, and suitably loading the pharmaceutical formulation into a syringe to form a pre-filled syringe.

Suitably, the pharmaceutical formulations described herein are prepared in sterile water, or are resuspended in sterile water for injection at the desired volume.

In exemplary embodiments, the pharmaceutical formulations have a volume of about 0.1 mL to about 20.0 mL, more suitably about 0.5 mL to about 15.0 mL, about 0.5 mL to about 12.0 mL, about 1.0 mL to about 10.0 mL, about 1.0 mL to about 5.0 mL, about 1.0 mL to about 2.0 mL or about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, about 1.5 mL, about 1.6 mL, about 1.7 mL, about 1.8 mL, about 1.9 mL, about 2.0 mL, about 2.1 mL, about 2.2 mL, about 2.3 mL, about 2.4 mL, about 2.5 mL, about 2.6 mL, about 2.7 mL, about 2.8 mL, about 2.9 mL, or about 3.0 mL, including any ranges or values within these ranges.

While in suitable embodiments, the pharmaceutical formulations described herein are liquid formulations, i.e., pharmaceutical formulations prepared in sterile water or water for injection (WFI), the pharmaceutical formulations can also be frozen formulations or previously lyophilized formulations.

The present invention also provides a lyophilized cake which is capable of being reconstituted using only sterile water into a formulation according to the invention as described herein. It will be understood that the ratio of antibody: ionic excipient will be the same in the lyophilized cake as in the post-lyophilized formulation. In one embodiment, the molar ratio of ionic excipient:antibody is in the range 450:1 to 40:1. Where the formulation has been lyophilized, the concentrations provided herein for the formulation are the post-reconstitution concentrations and thus are the concentrations in the so-called ‘drug product’. By way of example, if a half-reconstitution strategy is used (where half the volume of water removed during lyophilization is returned during reconstitution), then after reconstitution, the concentration of the antibody will be twice what it was prior to lyophilization i.e. twice what is was in the so-called pre-lyophilization ‘drug-substance’ composition. It will therefore be understood that the present invention further provides a composition capable of being lyophilized to form a lyophilized cake, wherein the lyophilized cake is capable of being reconstituted using only sterile water into a formulation according to the invention as described herein. Suitable reconstitution strategies will be known to those skilled in the art.

In embodiments, it is desirable to prepare frozen formulations by providing a liquid pharmaceutical formulation as described herein, and freezing the formulation under appropriate conditions. For example, the frozen formulations can be provided by freezing the liquid formulations to less than 0° C., more suitably to about −20° C., about −40° C., about −60° C., or suitably to about −80° C. The pharmaceutical formulations are also suitably prepared as liquid formulations and stored at about 2° C. to about 8° C., or about 2° C., about 3° C., about 4° C., about 5° C., about 6° C., about 7° C. or about 8° C.

Suitable protocols and methods for preparing lyophilized pharmaceutical formulations from liquid and/or frozen formulations are known in the art.

### Stability of Formulations

In exemplary embodiments, the formulations described herein are stable for extended periods of storage at room temperature or at a temperature range of about 2° C. to about 8° C., suitably about 5° C. As used herein, room temperature is generally in the range of about 22° C. to about 25° C. Suitably the pharmaceutical formulations are stable after storage at about 2° C. to about 8° C. (e.g. 5° C.) for at least six (6) months. As used herein, the term “stable” for a period of storage (or “stability”) is used to indicate that the monoclonal antibody, suitably IgG4 monoclonal antibody, pharmaceutical formulations resist aggregation, degradation, half antibody formation, and/or fragmentation. The stability of the monoclonal antibodies can be assessed by degrees of aggregation, degradation, half antibody formation or fragmentation, as measured by high performance size exclusion chromatography (HPSEC), static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference.

The overall stability of a pharmaceutical formulation comprising monoclonal antibodies can be assessed by various immunological assays including, for example, ELISA and radioimmunoassay using isolated antigen molecules.

The phrase “low to undetectable levels of aggregation” as used herein refers to pharmaceutical formulations containing no more than about 5%, no more than about 4%, no more than about 3%, no more than about 2%, no more than about 1%, or no more than about 0.5% aggregation by weight of protein as measured by high performance size exclusion chromatography (HPSEC) or static light scattering (SLS) techniques. Suitably, the pharmaceutical formulations exhibit ≤5.0% aggregation, more suitably ≤4.0% aggregation, ≤3.0% aggregation, ≤2.0% aggregation, ≤1.0% aggregation, or 0.5% aggregation. Suitably, the liquid pharmaceutical formulations and/or frozen pharmaceutical formulations exhibit ≤5.0% aggregation, more suitably ≤4.0% aggregation, ≤3.0% aggregation, ≤2.0% aggregation, ≤1.0% aggregation, or 0.5% aggregation.

The term “low to undetectable levels of fragmentation” as used herein refers to pharmaceutical formulations containing equal to or more than about 80%, about 85%, about 90%, about 95%, about 98%, or about 99% of the total monoclonal antibody, for example, in a single peak as determined by HPSEC, or reduced Capillary Gel Electrophoresis (rCGE), representing the non-degraded monoclonal antibody, or a non-degraded fragment thereof, and containing no other single peaks having more than about 5%, more than about 4%, more than about 3%, more than about 2%, more than about 1%, or more than about 0.5% of the total monoclonal antibody. Fragmentation may be measured suitably in IgG4 monoclonal antibodies. Without wishing to be bound by theory, it is thought that decreased self-aggregation is due to improved colloidal stability, as evidenced by increased kD value. In exemplary embodiments, the formulations described herein have reduced opalescence and decreased phase separation as detected by visual observation, light scattering, nephelometry or turbidimetric methods.

Further embodiments, features, and advantages of the embodiments, as well as the structure and operation of the various embodiments, are described in detail below with reference to accompanying drawings.

### EXAMPLES

### Example 1—IgG4 Formulations

The formulation challenge of antibodies with low or neutral pI values is that when these antibodies are formulated at a pH outside the range of 5.5 to 7.5 (as generally needed to increase protein charge and colloidal stability), additional instabilities are observed. At acidic pH an increased rate of fragmentation, reduced conformational stability and increased aggregation are observed. At basic pH the potential for increased oxidation, deamidation and fragmentation and incompatibility with glass containers are present. IgG4 antibodies are particularly useful for studying such instabilities since IgG4 antibodies typically have low or neutral pI values.

The following details the methods used to develop the formulations described herein that have optimized the storage stability of monoclonal antibodies that possess a neutral pI, and also overcomes the neutral pH physical instabilities mentioned above.

As described herein, the formulations have a combination of neutral pH and an ionic excipient such as a salt. Optionally a sugar may also be used in the formulations and this has been shown to provide further improvements in some cases.

The sugar (sucrose and trehalose are examples) can (in combination with an ionic excipient) further increase conformational stability; and the ionic excipient (sodium chloride, lysine hydrochloride and arginine hydrochloride for example) increases the colloidal stability of the monoclonal antibody molecules. Furthermore, the neutral pH (about pH 6) of the formulations also minimizes the acidic and basic degradation pathways described above. These formulations provide superior overall storage stability for these monoclonal antibodies. Other suitable components of the formulations are a buffer with a neutral pKa (e.g., NaAC, histidine HCl, sodium phosphate).

As described herein, and listed in Table 1, CAM-3001 (an anti-GM-CSFRα monoclonal antibody), two anti-IL-13 monoclonal antibodies, and an anti-C5/C5a monoclonal antibody were chosen for this study as they are IgG4's with neutral pI values.

TABLE 1
IgG4 Molecules Studied

| Molecule | Type | pI |
|---|---|---|
| CAM-3001 (anti- | IgG4 | 6.7 |
| GM-CSF-Rα) | | |
| anti-IL13 mAb A | IgG4 | 7.4 |
| (CAT-354) | | |
| anti-C5/C5a mAb | IgG4-P | 6.8-7.3 |
| (MEDI7814) | | |
| anti-IL-13 mAb B | IgG4-P | 6.6 |

CAT-354 is a human antibody of the IgG4 subclass that specifically binds the human interleukin 13 (IL-13), blocking interactions with the IL-13 receptor. The DNA and derived amino-acid sequences of the light chain and heavy chain for CAT-354 are provided in SEQ ID NOs: 1-6, respectively. The CAT-354 molecule is a human monoclonal IgG4 (lambda light-chain) antibody with a molecular weight of approximately 147,000 Daltons (Da) (including oligosaccharides). The antibody is composed of two identical heavy chains of approximately 49,500 Da each, and two identical light chains of approximately 22,500 Da each. CAT-354 contains fucosylated biantennary complex and high mannose N-linked carbohydrates attached to each heavy chain at Asn-299. The average size of the oligosaccharide moiety is approximately 1,650 Da per heavy chain.

CAM-3001 is an antibody which binds to Granulocyte Macrophage Colony Stimulating Factor Receptor alpha (GM-CSFRα). CAM-3001 is disclosed in International Patent Application Publication WO 2007/110631, the disclosure of which is incorporated by reference herein. The heavy chain CDRs (HCDRs) of CAM-3001 are disclosed as SEQ ID NOs: 53-55 in WO 2007/110631. The light chain CDRs (LCDRs) of CAM-3001 are disclosed as SEQ ID NOs: 58-60 in WO 2007/110631. The heavy chain variable region (VH) of CAM-3001 is disclosed as SEQ ID NO: 52 in WO 2007/110631. The light chain variable region (VL) of CAM-3001 is disclosed as SEQ ID NO: 218 in WO 2007/110631. The VH and VL sequences of CAM-3001 are shown in SEQ ID NO: 11, and SEQ ID NO: 12, respectively.

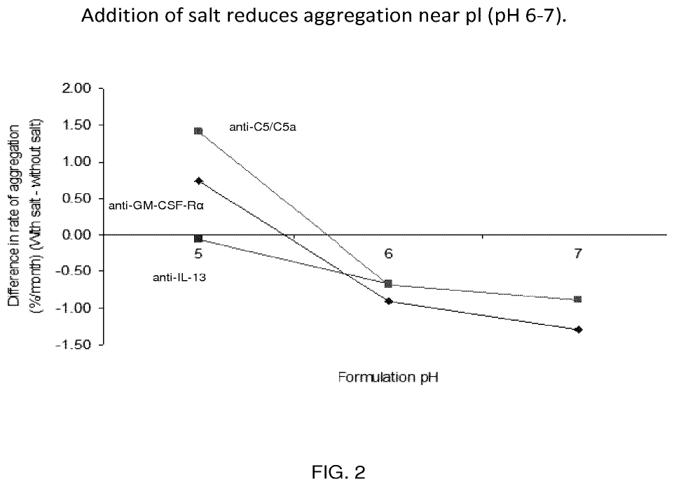

As shown in FIG. 2, in the presence of salt at a concentration of 100 mM, the aggregation rate increases when the molecule is at pH 5, but the aggregation rate is reduced when the molecule is at pH 6 and pH 7.

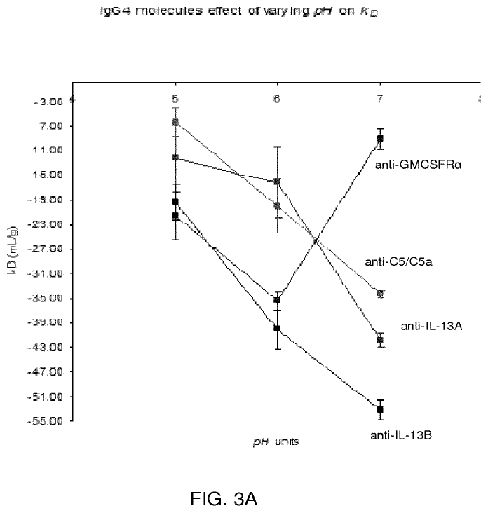

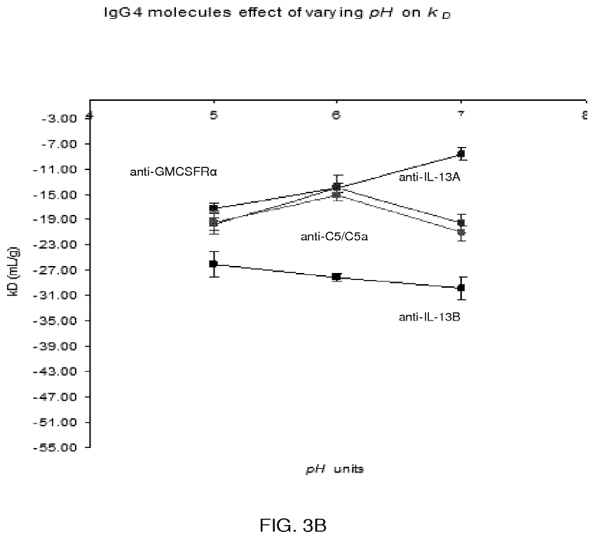

The correlation between solution properties of monoclonal antibodies with the diffusion interaction parameter (k_{D}) was examined by testing a combination of conformation stability and colloidal stability in order to achieve the most stable formulation. The k_{D} was measured as an indication of colloidal stability, while differential scanning calorimetry (DSC) was examined as a measure of conformational stability. FIG. 3 shows the k_{D} at varying pH for the monoclonal antibodies studied. FIG. 3B shows the k_{D} at varying pH for the monoclonal antibodies studied after adding salt at a concentration of 100 mM. At low ionic strength, the k_{D} generally becomes more negative with increasing pH. The addition of salt makes the k_{D} less negative for all molecules studied.

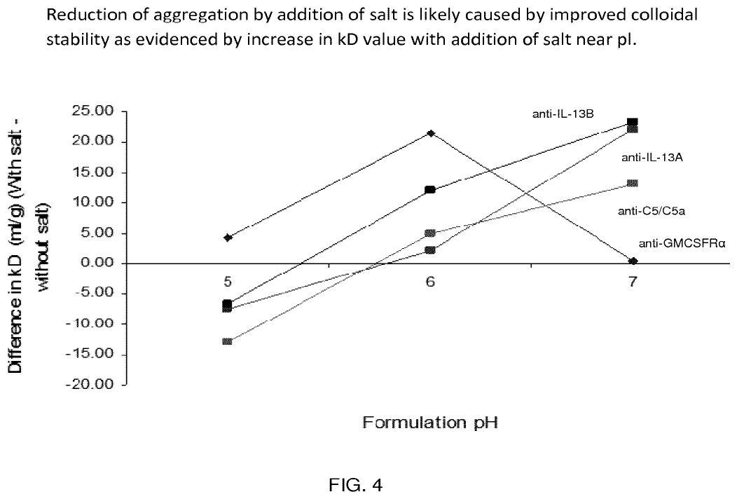

In the results depicted in FIG. 4, salt was added to the formulations at a concentration of 100 mM and the colloidal stability examined. Note generally a reduction in colloidal stability at pH 5, but an increase in colloidal stability at pH 6 and pH 7.

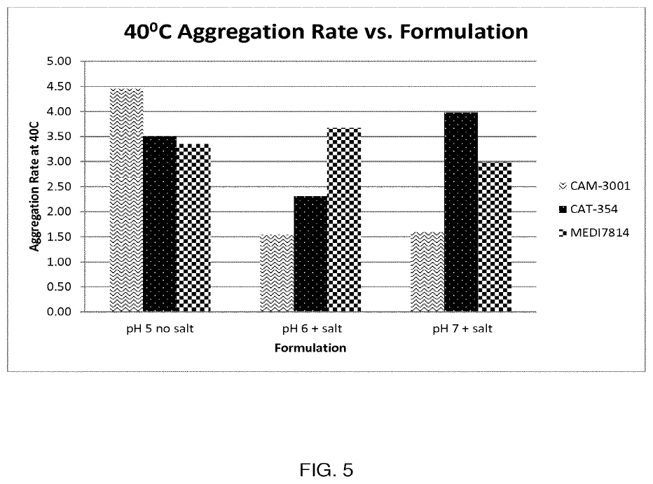

In the results depicted in FIG. 5, aggregation rate data at 40° C. was obtained for formulations with or without 100 mM salt. Formulations with salt near pI (pH 6 or 7) show reduced aggregation compared to formulation without salt away from pI.

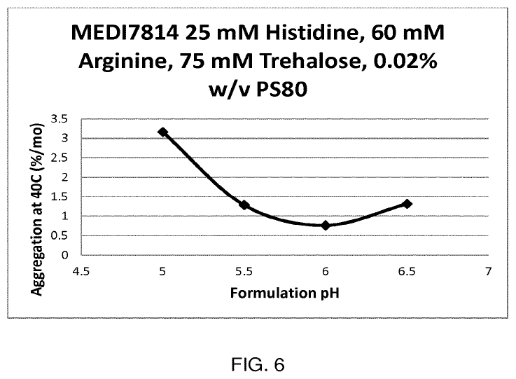

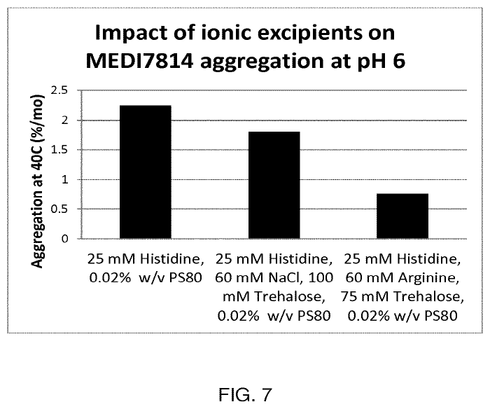

From the aggregation rate results depicted in FIGS. 6 and 7, it can be seen that charged amino acids can also act as ionic stabilizers near the molecule pI and may in some cases be more stabilizing than NaCl.

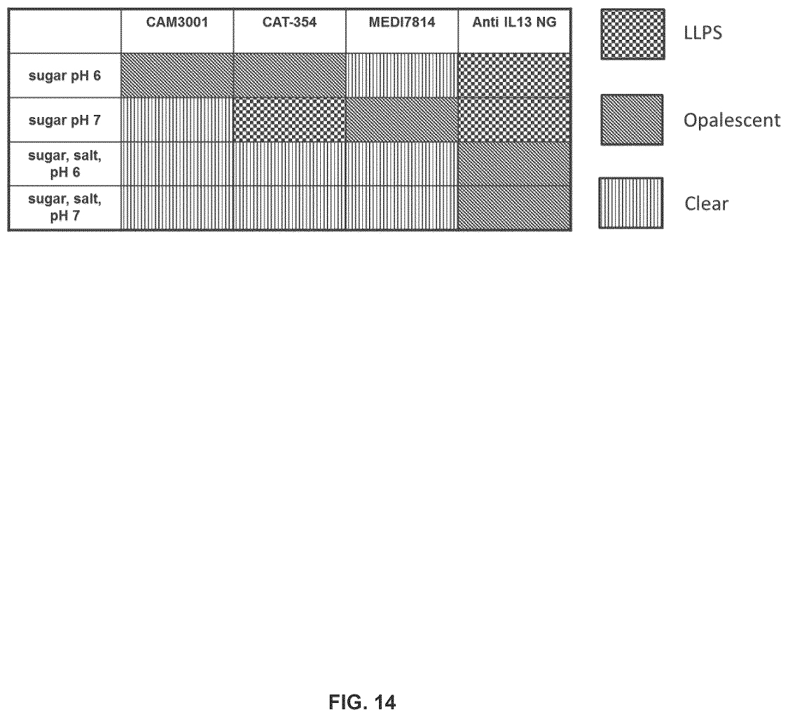

The addition of salt to a histidine-based buffer was examined to determine the effect on the stability of IgG4. The following buffer systems were examined: Control buffer (25 mM histidine, 7% sucrose, pH 6); Test buffer (25 mM histidine, 7% sucrose; 100 mM NaCl, pH 6) and the impact of salt on the appearance are summarized in FIG. 14.

### LLPS: Liquid-Liquid Phase Separation

As demonstrated above, the addition of salt reduced the rate of aggregation, and the rate of monomer loss, and improved the appearance of the formulations.

In summary, formulations comprising salt and with or without sugar have lower opalescence, mitigated phase separation in some instances, and provides as good or better stability when compared to formulations comprising sucrose alone.

Freeze-thaw studies showed that no significant changes in product quality, such as subvisible particles, were seen after three freeze-thaw cycles. In summary, the addition of both, salt and sugar, improved the stability of the IgG4 molecules, even when formulated at pHs near their pls.

### Example 2—I2G1 Formulation

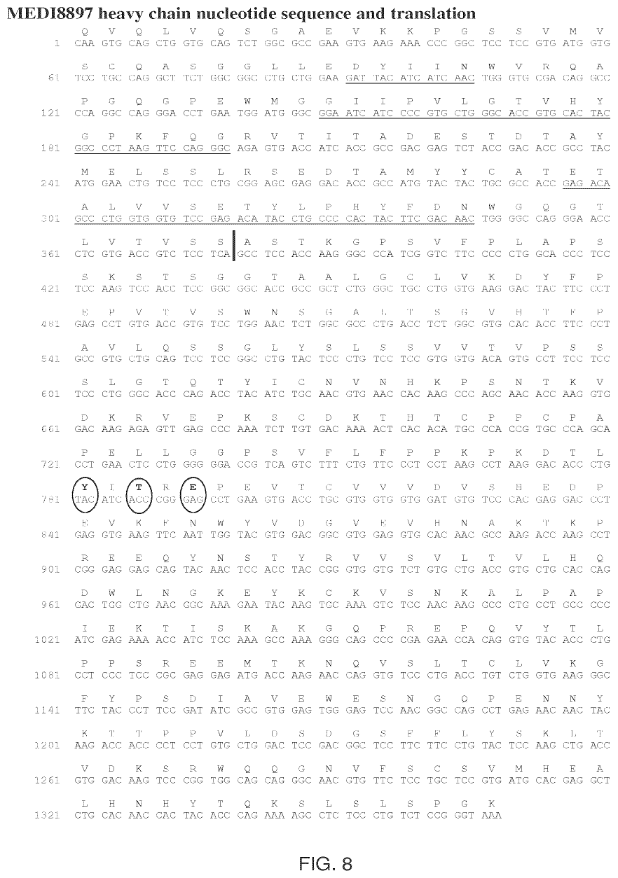

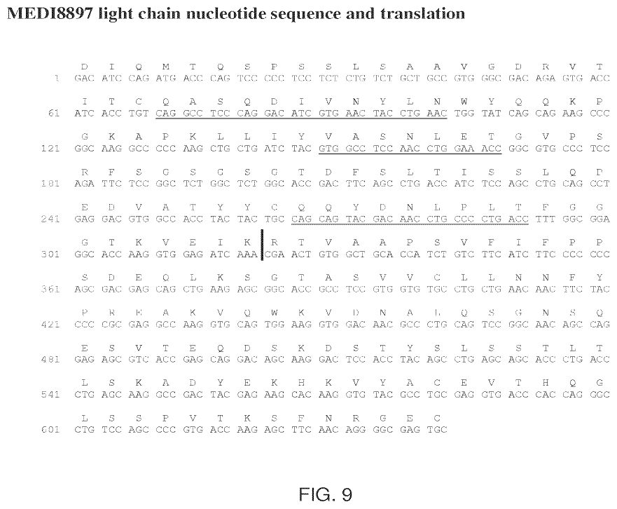

MEDI8897 is a human IgG1κ-YTE monoclonal antibody directed against RSV-F protein. Three amino acid substitutions (M252Y/S254T/T256E; called YTE) in the CH2 region of the Fc domain were introduced to increase the serum half-life of MEDI8897. Sequence information for MEDI8897 is provided in FIGS. 8 and 9. MEDI8897 pI was measured by cIEF to be 6.4-6.7 with the main peak at 6.4. The pI overlaps with the formulation buffer range (5.5-6.5) suggesting potential issues with manufacturing, formulation and storage stability.

MEDI8897 thermal stability was measured by differential scanning calorimetry. Tm1 was found to be 61° C. while Tm2 was 82° C. A Tm1 greater than 50° C. suggests that the molecule has acceptable colloidal stability.

### Stability Summary

Upon receipt of MEDI8897 in a standard buffer (25 mM Histidine, 7% sucrose, pH 6.0), phase separation was observed at 2 to 8° C. The supernatant layer had a protein concentration of 75 mg/ml while the bottom layer was 125 mg/ml. Upon equilibration at 25° C. the two distinct phases disappeared and only one single phase was observed. The phase separation at 2 to 8° C. was thought to be due to the pI of MEDI8897 which is close to the formulation pH of 6.0. A scouting study was initiated to find a more appropriate formulation buffer for MEDI8897 stability assessment, targeting a condition which maintained solubility and prevented phase separation of MEDI8897 at 100 mg/ml.

Formulating in the standard buffer (25 mM histidine, 7% sucrose) at pH's below 5.9 or above 6.7 mitigated phase separation. Addition of 75 mM NaCl to the standard buffer between pH 5.0 and 6.7 also mitigated phase separation. Finally, acetate and phosphate buffers at pH values away from the pI also mitigated phase separation. Based on these screening studies and previous knowledge of mAbs with pIs within the formulation space, an alternate buffer (25 mM His/HisHCl, 75 mM NaCl, 4% Sucrose, 0.02% PS80, pH 6.0) was selected for evaluation.

### kD Studies

For the first kD screen, all samples were evaluated in 25 mM Histidine pH 5.5 base buffer from 2-10 mg/ml at 25° C. This buffer was chosen in lieu of pH 6.0 because MEDI8897 is more soluble at pH 5.5, facilitating DLS measurements which are sensitive to insoluble particles. Ionic excipients including arginine-HCl, lysine-HCl and NaCl were evaluated at 10, 25, 50, 75 and 100 mM concentrations. In addition, proline, alanine, Na₂SO₄ and histidine were evaluated at the 100 mM concentration only. Finally, 2, 4, and 6% sucrose were evaluated to determine if sucrose influences protein-protein interactions. All conditions were compared to a buffer control (25 mM Histidine pH 5.5).

The control samples showed distinct protein-protein interactions, with the hydrodynamic radius increasing from 6.2 to 7.8 nm from 2-10 mg/ml. Arginine-HCl, lysine-HCl and NaCl showed reduction of protein-protein interactions (PPI) starting at 25 mM concentrations as evidenced by no increase in hydrodynamic size over the 2-10 mg/ml concentration range. No additional effects were seen between 25 and 100 mM. At 100 mM concentration, proline and alanine showed PPI similar to the control while Na₂SO₄ and Histidine mitigated PPI. Finally, sucrose concentration showed no impact on PPI. This data illustrates that charged excipients (Arg-HCl, Lys-HCl, Histidine and Na₂SO₄) mitigate protein-protein interactions while neutral excipients (sucrose, proline, alanine) do not mitigate PPI. Therefore, addition of ionic excipients at pH 5.5 reduced phase separation at 100 mg/ml.

### 40° C. Stability Evaluation

Based on kD screening, several conditions were selected for 40° C. stability evaluation. Table 3 summarizes the formulation conditions and 1 month degradation rates seen at 40° C.

TABLE 3
40° C. Stability Rates, Formulation Screen 1- Excipient Screening

| | | Conc | | | |
|---|---|---|---|---|---|
| Number | Excipient | (mM) | % Mon/mo | % Agg/mo | % Frag/mo |
| 1 | NaCl | 25 | −5.9 | 4.2 | 1.8 |
| 2 | NaCl | 75 | −6.1 | 4.1 | 1.9 |
| 3 | NaCl | 95 | −5.4 | 3.5 | 1.9 |
| 4 | NaCl | 120 | −5.4 | 3.5 | 1.9 |
| 5 | Arg-HCl | 25 | −5.4 | 3.5 | 1.8 |
| 6 | Arg-HCl | 75 | −4.8 | 2.8 | 2.0 |
| 7 | Arg-HCl | 95 | −4.5 | 2.6 | 1.9 |
| 8 | Arg-HCl | 120 | −4.8 | 2.8 | 2.0 |
| 9 | Lys-HCl | 25 | −5.7 | 3.9 | 1.9 |
| 10 | Lys-HCl | 75 | −5.0 | 2.7 | 2.3 |
| 11 | Lys-HCl | 95 | −5.1 | 3.1 | 2.0 |
| 12 | Lys-HCl | 120 | −4.9 | 2.9 | 2.0 |

Base buffer for this study was 25 mM Histidine pH 6.0

This study illustrates that arginine and lysine are more stabilizing than NaCl. In addition, 75 mM and above appears to stabilize against aggregation. Based on this study, arginine was selected as the most stabilizing lyo-friendly excipient and was moved forward to the next set of studies.

### Drug Product Stability on Final Lyo Cycle/Representative Material

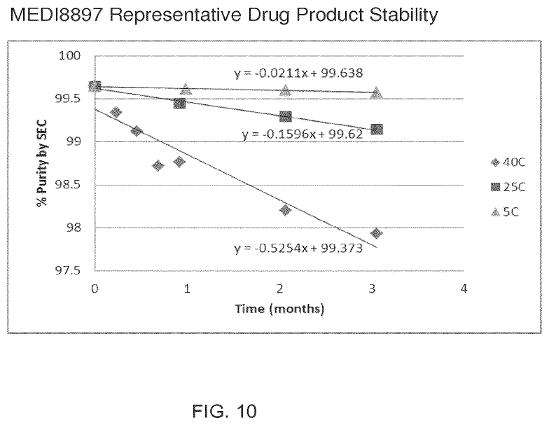

Stability of the drug product was evaluated. Three months of data was collected for the post reconstitution formulation of 100 mg/ml in 30 mM L-histidine/L-histidine hydrochloride monohydrate, 80 mM L-arginine hydrochloride, 120 mM sucrose, 0.04% (w/v) polysorbate 80, pH 6.0. Results are shown in FIG. 10. Storage at 2-8° C. showed virtually no change during the 3 month period, confirming the suitability of the formulation and lyo cycle for clinical use. These data thus demonstrate that the formulation provides appropriate stability and solubility and is suitable for first time in human clinical use.

TABLE 4
Drug Product Stability 3 Month Data Summary

| | HIAC | HIAC | Bio- | Recon | | |
|---|---|---|---|---|---|---|
| Temperature | (≥10 μm) | (≥25 μm) | assay | Time | VI | KF |
| 2-8° C. | 216 | 108 | 97% | 2 min | <STD1 | 1.3% |
| 25° C. | 522 | 90 | 97% | 3 min | <STD1 | 1.4% |
| 40° C. | 126 | 0 | 90% | 3 min | <STD2 | 1.7% |

### Example 3—IgG4 Formulation

Formulations of antibody MEDI578 were tested at pH6. MEDI578 is an IgG4 monoclonal antibody having a pI of 6.3 to 6.8.

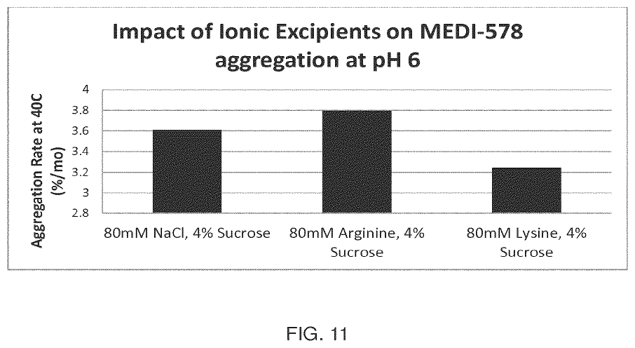

Results are shown in FIG. 11.

### Example 4—IgG4 Formulation

All formulations that were tested contained 100 mg/ml MEDI578, 20 mM histidine pH 6.5 without polysorbate. MEDI578 is an IgG4 monoclonal antibody having a pI of 6.3 to 6.8.

TABLE 5
Formulation information

| Formulation | | | Osmo | | |
|---|---|---|---|---|---|
| # | Excipients | Tg′ | (mOsm/kg) | Opalescence | Particles |
| 1 | 9% Sucrose | −25.8 | 387 | 4 | 2 |
| 2 | 9% Trehalose | −23.6 | 341 | 4 | 2 |
| 3 | 275 mM Mannitol | −28.2 | 331 | 5 | 2 |
| 4 | 125 mM Lys/35 mM NaCl | −30.1 | 361 | 3 | 1 |
| 5 | 20 mM Lys/8% sucrose | −25.7 | 392 | 3 | 1 |
| 6 | 50 mM Lys/6% sucrose | −26.4 | 379 | 3 | 1 |
| 7 | 80 mM Lys/4% sucrose | −27.3 | 326 | 3 | 1 |
| 8 | 80 mM NaCl/4% sucrose | −26 | 365 | 3 | 1 |
| 9 | 80 mM Arg/4% sucrose | −24.4 | 363 | 3 | 1 |
| 10 | 150 mM Mannitol/4% sucrose | −27.8 | 379 | 5 | 2 |

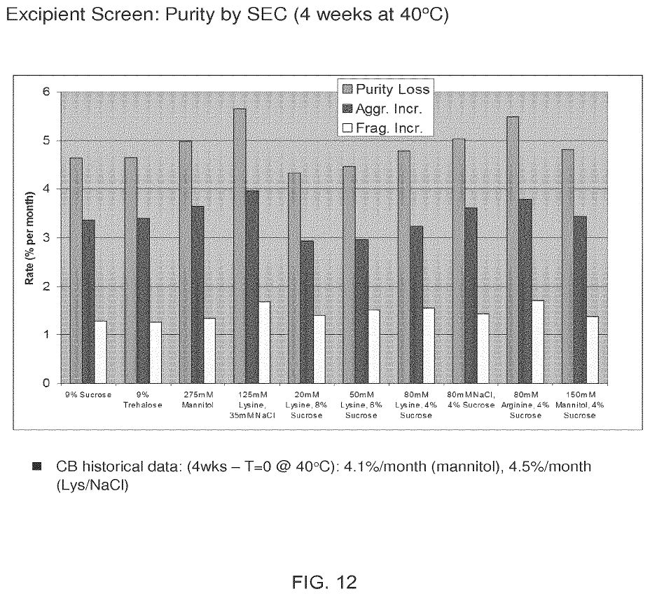

FIG. 12 shows purity by size-exclusion chromatography.

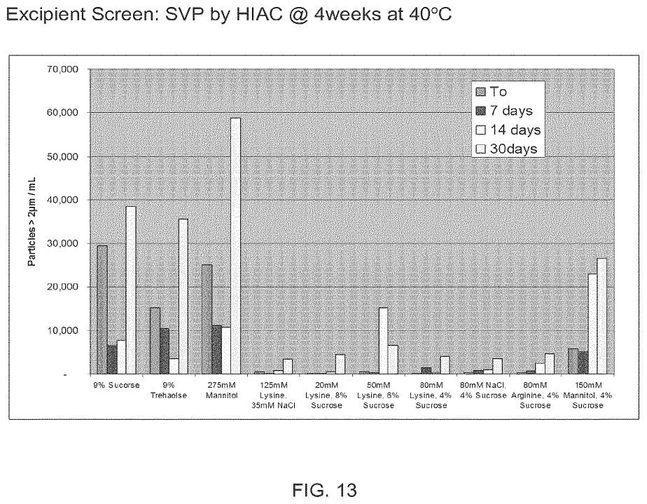

FIG. 13 shows a reduction in subvisible particles in formulations containing ionic excipients under thermal stress conditions. Colloidal stability is improved.

All documents, patents, journal articles and other materials cited in the present application are hereby incorporated by reference.

Although the present invention has been fully described in conjunction with several embodiments thereof with reference to the accompanying drawings, it is to be understood that various changes and modifications can be apparent to those skilled in the art. Such changes and modifications are to be understood as included within the scope of the present invention as defined by the appended claims, unless they depart there from.

1. A formulation comprising:
i. a monoclonal antibody; and
ii. an ionic excipient;
wherein the monoclonal antibody is present at a concentration of about 50 mg/ml or greater and the ionic excipient is present at a concentration of about 50 to about 150 mM and the formulation has a pH of 5.5 to 7.5, and wherein the monoclonal antibody comprises an anti-GM-CSF-Rα monoclonal antibody.

2. A formulation comprising:
i. a monoclonal antibody; and
ii. an ionic excipient;
wherein the monoclonal antibody is present at a concentration of about 50 mg/ml or greater and the ionic excipient is present at a concentration of about 50 to about 150 mM and the formulation has a pH of 5.5 to 7.5; and wherein the aggregation rate of the monoclonal antibody in the formulation is reduced compared to the aggregation rate of the same antibody in the same formulation but without an ionic excipient, and wherein the monoclonal antibody comprises an anti-GM-CSF-Rα monoclonal antibody.

3. The formulation according to claim 1, wherein the monoclonal antibody has a pI in the range of pH 5.5 to pH 7.5.

4.-6. (canceled)

7. The formulation according to claim 1, wherein the monoclonal antibody is present in the formulation at a concentration of about 100 mg/ml to about 200 mg/ml.

8. (canceled)

9. The formulation according to claim 1, wherein the formulation has a pH in the range of about pH 5.5 to about pH 6.5.

10.-12. (canceled)

13. The formulation according to claim 1, wherein the ionic excipient is a salt.

14.-16. (canceled)

17. The formulation according to claim 1, wherein the ionic excipient is present at a concentration of about 75 mM to about 100 mM.

18. (canceled)

19. The formulation according to claim 1, wherein the formulation further comprises a sugar.

20.-21. (canceled)

22. The formulation according to claim 19, wherein the sugar is present at a concentration of about 100 mM to about 140 mM.

23. (canceled)

24. The formulation according to claim 1, wherein the formulation further comprises one or more buffers.

25.-26. (canceled)

27. The formulation according to claim 24, wherein the one or more buffers is present at a concentration of about 10 mM to about 50 mM.

28. (canceled)

29. The formulation according to claim 1, wherein the formulation further comprises a surfactant.

30.-31. (canceled)

32. The formulation according to claim 29, wherein the surfactant is present in the formulation at a concentration from about 0.02% (w/v) to about 0.07% (w/v).

33. (canceled)

34. The formulation according to claim 1, wherein the formulation further comprises one or more additional excipients, including for example, one or more sugars, salts, amino acids, polyols, chelating agents, emulsifiers and/or preservatives.

35.-37. (canceled)

38. A formulation comprising 150 mg/ml anti-GM-CSF-Rα IgG4 antibody, 50 mM sodium acetate/acetic acid, 106 mM trehalose dehydrate, 70 mM sodium chloride, and 0.05% (w/v) Polysorbate 80, wherein the formulation has a pH of pH 5.8.

39. The formulation according to claim 1, wherein the antibody has the 6 CDR sequences of CAM-3001.

40. The formulation according to claim 1, wherein the antibody has the VH and VL sequences of CAM-3001.

41.-43. (canceled)

44. A method of treating a disease in a subject comprising administering the formulation according to claim 1 to the subject.

45. A lyophilized cake capable of being reconstituted using only sterile water into a formulation as defined in claim 1.

46. A formulation capable of being lyophilized to form a lyophilized cake, wherein the lyophilized cake is capable of being reconstituted using only sterile water into a formulation as defined in claim 1.


SEQUENCE LISTING (10 sequences)

SEQ ID NO: 1 - DNA, 1074 bp, synthetic construct
  misc_feature 1..1074: note = Coding strand for CAT-354 light chain DNA sequence
  source 1..1074: mol_type = other DNA
tcagatcgcctggagacgccatccacgctgttttgacctccatagaagacaccgggaccgatccagcctccgcggccgggaacggtgcattggaacgcggattccccgtgccaagagtgactcaccgtccttgacacgaagctattcgaactacagttactgagcacacaggacctcaccatgggatggagctgtatcatcctcttcttggtagcaacagctacaggtgtccactcctcctatgtgctgactcagccaccctcggtgtcagtggccccaggaaagacggccaggattacctgtgggggaaacatcattggaagtaaacttgtacactggtaccagcagaagccaggccaggcccctgtgctggtcatctatgatgatggcgaccggccctcagggatccctgagcgattctctggctccaactctgggaacacggccaccctgaccatcagcagggtcgaggccggggatgaggccgactattattgtcaggtgtgggatactggtagtgatcccgtggtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataggaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttc

SEQ ID NO: 2 - DNA, 1074 bp, synthetic construct
  misc_feature 1..1074: note = Complementary strand of CAT-354 light chain DNA sequence
  source 1..1074: mol_type = other DNA
agtctagcggacctctgcggtaggtgcgacaaaactggaggtatcttctgtggccctggctaggtcggaggcgccggcccttgccacgtaaccttgcgcctaaggggcacggttctcactgagtggcaggaactgtgcttcgataagcttgatgtcaatgactcgtgtgtcctggagtggtaccctacctcgacatagtaggagaagaaccatcgttgtcgatgtccacaggtgaggaggatacacgactgagtcggtgggagccacagtcaccggggtcctttctgccggtcctaatggacaccccctttgtagtaaccttcatttgaacatgtgaccatggtcgtcttcggtccggtccggggacacgaccagtagatactactaccgctggccgggagtccctagggactcgctaagagaccgaggttgagacccttgtgccggtgggactggtagtcgtcccagctccggcccctactccggctgataataacagtccacaccctatgaccatcactagggcaccataagccgcctccctggttcgactggcaggatccagtcgggttccgacgggggagccagtgagacaagggcgggaggagactcctcgaagttcggttgttccggtgtgaccacacagagtattcactgaagatgggccctcggcactgtcaccggaccttccgtctatcgtcggggcagttccgccctcacctctggtggtgtgggaggtttgtttcgttgttgttcatgcgccggtcgtcgatagactcggactgcggactcgtcaccttcagggtgtcttcgatgtcgacggtccagtgcgtacttccctcgtggcacctcttctgtcaccggggatgtcttacaagtatccttaagtaactagtattagtcggtatggtgtaaacatctccaaaatgaacgaaattttttggagggtgtggagggggacttggactttgtattttacttacgttaacaacaacaattgaacaaataacgtcgaatattaccaatgtttatttcgttatcgtagtgtttaaagtgtttatttcgtaaaaaaag

SEQ ID NO: 3 - protein, 233 aa, synthetic construct
  REGION 1..233: note = CAT-354 light chain
MGWSCIILFLVATATGVHSSYVLTQPPSVSVAPGKTARITCGGNIIGSKLVHWYQQKPGQAPVLVIYDDGDRPSGIPERFSGSNSGNTATLTISRVEAGDEADYYCQVWDTGSDPVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 4 - DNA, 1937 bp, synthetic construct
  misc_feature 1..1937: note = Coding sequence for CAT-354 heavy chain DNA sequence
  source 1..1937: mol_type = other DNA
tcagatcgcctggagacgccatccacgctgttttgacctccatagaagacaccgggaccgatccagcctccgcggccgggaacggtgcattggaacgcggattccccgtgccaagagtgactcaccgtccttgacacgaagcttactgagcacacaggacctcaccatgggatggagctgtatcatcctcttcttggtagcaacagctacaggtgtccactcccaagtgcagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcagcgctaataatggcgacacaaattatggacaggaattccagggcagagtcaccatgaccacagatacatccacgagcacagcctacatggagttgaggagcctgagatctgacgacacggccgtttattactgtgcgagagactccagcagcagctgggctcgctggtttttcgatctctggggccgggggacactggtcaccgtctcgagtgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtcgacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctggctcgaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctt

SEQ ID NO: 5 - DNA, 1937 bp, synthetic construct
  misc_feature 1..1937: note = Complementary strand for CAT-354 heavy chain DNA sequence
  source 1..1937: mol_type = other DNA
agtctagcggacctctgcggtaggtgcgacaaaactggaggtatcttctgtggccctggctaggtcggaggcgccggcccttgccacgtaaccttgcgcctaaggggcacggttctcactgagtggcaggaactgtgcttcgaatgactcgtgtgtcctggagtggtaccctacctcgacatagtaggagaagaaccatcgttgtcgatgtccacaggtgagggttcacgtcgaccacgtcagaccccgactccacttcttcggaccccggagtcacttccagaggacgttccgaagaccaatgtggaaatgtttaataccagagtcgacccacgctgtccggggacctgttcccgaactcacctaccctacctagtcgcgattattaccgctgtgtttaatacctgtccttaaggtcccgtctcagtggtactggtgtctatgtaggtgctcgtgtcggatgtacctcaactcctcggactctagactgctgtgccggcaaataatgacacgctctctgaggtcgtcgtcgacccgagcgaccaaaaagctagagaccccggccccctgtgaccagtggcagagctcacgaaggtggttcccgggtaggcagaagggggaccgcgggacgaggtcctcgtggaggctctcgtgtcggcgggacccgacggaccagttcctgatgaaggggcttggccactgccacagcaccttgagtccgcgggactggtcgccgcacgtgtggaagggccgacaggatgtcaggagtcctgagatgagggagtcgtcgcaccactggcacgggaggtcgtcgaacccgtgcttctggatgtggacgttgcatctagtgttcgggtcgttgtggttccagctgttctctcaactcaggtttataccagggggtacgggtagtacgggtcgtggactcaaggacccccctggtagtcagaaggacaaggggggttttgggttcctgtgagagtactagagggcctggggactccagtgcacgcaccaccacctgcactcggtccttctggggctccaggtcaagttgaccatgcacctaccgcacctccacgtattacggttctgtttcggcgccctcctcgtcaagttgtcgtgcatggcacaccagtcgcaggagtggcaggacgtggtcctgaccgacttgccgttcctcatgttcacgttccagaggttgtttccggagggcaggaggtagctcttttggtagaggtttcggtttcccgtcggggctctcggtgtccacatgtgggacgggggtagggtcctcctctactggttcttggtccagtcggactggacggaccagtttccgaagatggggtcgctgtagcggcacctcaccctctcgttacccgtcggcctcttgttgatgttctggtgcggagggcacgacctgaggctgccgaggaagaaggagatgtcgtccgattggcacctgttctcgtccaccgtcctccccttacagaagagtacgaggcactacgtactccgagacgtgttggtgatgtgtgtcttctcggagagggacagagacccatttactcacggtcccggccgttcgggggcgaggggcccgagagccccagcgcgctcctacgaaccgtgcatggggcagatgtatgaagggtccgtgggtcgtacctttatttcgtgggtggtgacgggaccgagcttaagtaactagtattagtcggtatggtgtaaacatctccaaaatgaacgaaattttttggagggtgtggagggggacttggactttgtattttacttacgttaacaacaacaattgaacaaataacgtcgaatattaccaatgtttatttcgttatcgtagtgtttaaagtgtttatttcgtaaaaaaagtgacgtaagatcaacaccaaacaggtttgagtagttacatagaa

SEQ ID NO: 6 - protein, 468 aa, synthetic construct
  REGION 1..468: note = CAT-354 heavy chain
MGWSCIILFLVATATGVHSQVQLVQSGAEVKKPGASVKVSCKASGYTFTNYGLSWVRQAPGQGLEWMGWISANNGDTNYGQEFQGRVTMTTDTSTSTAYMELRSLRSDDTAVYYCARDSSSSWARWFFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 456 aa, synthetic construct
  REGION 1..456: note = MEDI8897 heavy chain
QVQLVQSGAEVKKPGSSVMVSCQASGGLLEDYIINWVRQAPGQGPEWMGGIIPVLGTVHYGPKFQGRVTITADESTDTAYMELSSLRSEDTAMYYCATETALVVSETYLPHYFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 1368 bp, synthetic construct
  misc_feature 1..1368: note = MEDI8897 heavy chain
  source 1..1368: mol_type = other DNA
caagtgcagctggtgcagtctggcgccgaagtgaagaaacccggctcctccgtgatggtgtcctgccaggcttctggcggcctgctggaagattacatcatcaactgggtgcgacaggccccaggccagggacctgaatggatgggcggaatcatccccgtgctgggcaccgtgcactacggccctaagttccagggcagagtgaccatcaccgccgacgagtctaccgacaccgcctacatggaactgtcctccctgcggagcgaggacaccgccatgtactactgcgccaccgagacagccctggtggtgtccgagacatacctgccccactacttcgacaactggggccagggaaccctcgtgaccgtctcctcagcctccaccaagggcccatcggtcttccccctggcaccctcctccaagtccacctccggcggcaccgccgctctgggctgcctggtgaaggactacttccctgagcctgtgaccgtgtcctggaactctggcgccctgacctctggcgtgcacaccttccctgccgtgctgcagtcctccggcctgtactccctgtcctccgtggtgacagtgccttcctcctccctgggcacccagacctacatctgcaacgtgaaccacaagcccagcaacaccaaggtggacaagagagttgagcccaaatcttgtgacaaaactcacacatgcccaccgtgcccagcacctgaactcctggggggaccgtcagtctttctgttccctcctaagcctaaggacaccctgtacatcacccgggagcctgaagtgacctgcgtggtggtggatgtgtcccacgaggaccctgaggtgaagttcaattggtacgtggacggcgtggaggtgcacaacgccaagaccaagcctcgggaggagcagtacaactccacctaccgggtggtgtctgtgctgaccgtgctgcaccaggactggctgaacggcaaagaatacaagtgcaaagtctccaacaaggccctgcctgcccccatcgagaaaaccatctccaaagccaaagggcagccccgagaaccacaggtgtacaccctgcctccctcccgcgaggagatgaccaagaaccaggtgtccctgacctgtctggtgaagggcttctacccttccgatatcgccgtggagtgggagtccaacggccagcctgagaacaactacaagaccacccctcctgtgctggactccgacggctccttcttcctgtactccaagctgaccgtggacaagtcccggtggcagcagggcaacgtgttctcctgctccgtgatgcacgaggctctgcacaaccactacacccagaaaagcctctccctgtctccgggtaaa

SEQ ID NO: 9 - protein, 214 aa, synthetic construct
  REGION 1..214: note = MEDI8897 light chain
DIQMTQSPSSLSAAVGDRVTITCQASQDIVNYLNWYQQKPGKAPKLLIYVASNLETGVPSRFSGSGSGTDFSLTISSLQPEDVATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 642 bp, synthetic construct
  misc_feature 1..642: note = MEDI8897 light chain
  source 1..642: mol_type = other DNA
gacatccagatgacccagtccccctcctctctgtctgctgccgtgggcgacagagtgaccatcacctgtcaggcctcccaggacatcgtgaactacctgaactggtatcagcagaagcccggcaaggcccccaagctgctgatctacgtggcctccaacctggaaaccggcgtgccctccagattctccggctctggctctggcaccgacttcagcctgaccatctccagcctgcagcctgaggacgtggccacctactactgccagcagtacgacaacctgcccctgacctttggcggaggcaccaaggtggagatcaaacgaactgtggctgcaccatctgtcttcatcttcccccccagcgacgagcagctgaagagcggcaccgcctccgtggtgtgcctgctgaacaacttctacccccgcgaggccaaggtgcagtggaaggtggacaacgccctgcagtccggcaacagccaggagagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaacaggggcgagtgc